(19) United States
(12) Patent Application Publication
(10) Pub. No.: US 2026/0123874 A1
(43) Pub. Date: May 7, 2026

(54) ARRHYTHMIA CLASSIFICATION METHOD USING ECG SIGNALS, APPARATUS THEREOF, DEVICE AND MEDIUM

(71) Applicants: Peking Union Medical College, Beijing (CN); University of Science and Technology Beijing, Beijing (CN)

(72) Inventors: Meihua PIAO, Beijing (CN); Hongzhen CUI, Beijing (CN); Yunfeng PENG, Beijing (CN); Haoming MA, Beijing (CN); Aoqi WANG, Beijing (CN); Xingyi TANG, Beijing (CN); Sijia LI, Beijing (CN); Longhao ZHANG, Beijing (CN); Hefei HAO, Beijing (CN)

(21) Appl. No.: 19/024,528

(22) Filed: Jan. 16, 2025

(30) Foreign Application Priority Data
Nov. 1, 2024 (CN) 202411554086.2

(51) Int. Cl. A61B 5/364; A61B 5/36; A61B 5/366; G16H 10/60

(52) U.S. Cl. CPC A61B 5/364; A61B 5/36; A61B 5/366; G16H 10/60

(57) ABSTRACT

Provided is an arrhythmia classification method using ECG signals, an apparatus thereof, a device and a medium. The method includes: acquiring ECG data and general patient data; constructing the first and second BERT models; inputting the ECG data and the general patient data into the first and second BERT models to obtain the first and second semantic information vector; constructing the first and second tower layers; inputting the first and second semantic information vectors into the first and second tower layers respectively, to obtain the first and second feature vectors; calculating a correlation coefficient based on the first and second feature vectors; carrying out feature combination on the first and second feature vectors and the correlation coefficient to obtain a combined feature; constructing a classification model; and inputting the combined feature into the classification model to obtain the category of arrhythmia classification.

### CROSS-REFERENCE TO RELATED APPLICATION

This patent application claims the benefit and priority of Chinese Patent Application No. 2024115540862 filed with the China National Intellectual Property Administration on Nov. 1, 2024, the disclosure of which is incorporated by reference herein in its entirety as part of the present disclosure.

### TECHNICAL FIELD

The present disclosure relates to the field of arrhythmia classification, in particular to an arrhythmia classification method using Electrocardiograph (ECG) signals, an apparatus thereof, a device and a medium.

### BACKGROUND

With the introduction and development of computer neural networks and large models, various cardiovascular diseases are constantly being judged by the application of models such as deep learning, and certain research results have been achieved. However, it is difficult to classify and diagnose individual differences resulted from specific types of arrhythmia diseases. It is difficult to make regular judgments on the potential features of data through human beings, which is limited by deep learning and the generalization ability and accuracy of existing models in arrhythmia diseases.

The existing main techniques of diagnosing arrhythmia using ECG signals include traditional methods and machine learning-based methods. The traditional methods include manual analysis, rule or threshold definition, etc. Manual analysis needs manual interpretation from a cardiologist. By observing the shapes, the intervals and the amplitudes of P wave, QRS complex and T wave, screening and diagnosis are carried out according to experience. Manual analysis is time-consuming and labor-consuming, and relies on expert experience. There may be subjective errors or misjudgments. Manual analysis is not suitable for large-scale screening or diagnosis. When judging R-R interval, the method based on rules and thresholds automatically judges whether arrhythmia exists in the P-R interval. Obviously, in the case of individual differences, the thresholds or rules are inaccurate and lack of adaptability, which cannot ensure the processing of complex arrhythmia and cannot ensure the accuracy and robustness. According to the machine learning-based methods, features such as heart rate variability, waveform morphological features, waveform variation features, etc. are first extracted from ECG signals, and then the arrhythmia is classified and diagnosed by combining with classification algorithms, such as a support vector machine, a decision tree, a random forest, etc. The most serious defect of the process is that the process relies on experts in the field of cardiology, the sensitivity of feature selection and model performance to data distribution needs a lot of labeled data to be trained, and there is weak robustness to the diagnosis and judgment of abnormal unlabeled data.

Therefore, the present disclosure provides an arrhythmia classification method using ECG signals, an apparatus thereof, a device, a medium and a product.

### SUMMARY

The purpose of the present disclosure is to provide an arrhythmia classification method using ECG signals, an apparatus thereof, a device, a medium and a product, which can improve the accuracy of arrhythmia classification.

In order to achieve the above purposes, the present disclosure provides the following solution.

In a first aspect, the present disclosure provides an arrhythmia classification method using ECG signals, including:
acquiring Electrocardiograph (ECG) data and general patient data;
constructing a first Bidirectional Encoder Representations from Transformers (BERT) model and a second BERT model;
inputting the ECG data and the general patient data into the first BERT model and the second BERT model to obtain a first semantic information vector and a second semantic information vector;
constructing a first tower layer and a second tower layer;
inputting the first semantic information vector and the second semantic information vector into the first tower layer and the second tower layer, respectively, to obtain a first feature vector and a second feature vector;
calculating a correlation coefficient based on the first feature vector and the second feature vector;
carrying out feature combination on the first feature vector, the second feature vector and the correlation coefficient to obtain a combined feature;
constructing a classification model; and
inputting the combined feature into the classification model to obtain a category of arrhythmia classification.

Preferably, the general patient data includes an age, a sex, a height and a weight;
the ECG data includes general ECG data and lead data distribution, the general ECG data includes QRS duration, P-R interval, P-T interval, T interval, P interval, QRS wave, T wave, P wave, QRST wave and J point; and the lead data distribution includes a wave width and an amplitude.

Preferably, the first tower layer is any one of Long Short-Term Memory (LSTM), Gate Recurrent Unit (GRU), Bidirectional Long Short-Term Memory (BILSTM), Bidirectional Gate Recurrent Unit (BiGRU), Convolutional Neural Network (CNN), Recurrent Neural Network (RNN), Transformer and Attention;
the second tower layer is any one of LSTM, GRU, BILSTM, BIGRU, CNN, RNN, Transformer and Attention.

Preferably, the correlation coefficient is calculated based on the first feature vector and the second feature vector using any one of methods such as cosine similarity, Euclidean distance, Jaccard similarity coefficient, Pearson correlation coefficient, Manhattan distance, Spearman's rank correlation coefficient, angular similarity, cosine distance and Chebyshev distance.

Preferably, the correlation coefficient is calculated based on the first feature vector and the second feature vector specifically using the following formula:

$$cos θ = \frac{{\sum }_{i = 1}^{n} ( {v}_{i} \times {μ}_{i} )}{\sqrt{{\sum }_{i = 1}^{n} {( {v}_{i} )}^{2}} \times \sqrt{{\sum }_{i = 1}^{n} {( {μ}_{i} )}^{2}}} or cos θ = \frac{{V}_{1} {V}_{2}}{‖ {V}_{1} ‖ \times ‖ {V}_{2} ‖}$$

$$K = cos θ$$

wherein V₁={v₁, v₂, . . . , vₙ} represents the first feature vector, and V₂={μ₁, μ₂, . . . , μₙ} represents the second feature vector.

Preferably, the expression of the classification model is:

$$f ( x ) = {ω}^{T} φ ( x ) \text{?} b = sign ( {\sum }_{i = 1}^{n} {y}_{i} \text{?} K ( \text{?} ) + b )$$

$$\text{?} \text{indicates text missing or illegible when filed}$$

where ω represents a parameter matrix, i.e. a weight vector, T represents transposition calculation, φ(x) represents a nonlinear mapping function that maps input features into a high-dimensional space, b represents a bias term, n represents the number of patients, i represents an i-th patient, yᵢ represents a classification category of the i-th patient with arrhythmia, yⱼ represents a classification category of a j-th patient with arrhythmia, represents αᵢ Lagrange multiplier (a coefficient of support vector) corresponding to an i-th training sample, namely, the patient with arrhythmia, and K(xᵢ, yⱼ) represents a kernel function, which is used to calculate a similarity between input features.

In a second aspect, the present disclosure provides an arrhythmia classification apparatus using ECG signals, including:
a data acquiring module, configured to acquire Electrocardiograph (ECG) data and general patient data;
a BERT model constructing module, configured to construct a first BERT model and a second BERT model;
a semantic information vector determining module, configured to input the ECG data and the general patient data into the first BERT model and the second BERT model to obtain a first semantic information vector and a second semantic information vector;
a tower layer constructing module, configured to construct a first tower layer and a second tower layer;
a feature vector determining module, configured to input the first semantic information vector and the second semantic information vector into the first tower layer and the second tower layer, respectively, to obtain a first feature vector and a second feature vector;
a correlation coefficient calculating module, configured to calculate a correlation coefficient based on the first feature vector and the second feature vector;
a feature combining module, configured to carry out feature combination on the first feature vector, the second feature vector and the correlation coefficient to obtain a combined feature;
a classification model constructing module, configured to construct a classification model; and
a classifying module, which is configured to input the combined feature into the classification model to obtain a category of arrhythmia classification.

In a third aspect, the present disclosure provides a computer device, including: a memory, a processor and a computer program which is stored in the memory and is executable on the processor, wherein the processor executes the computer program to implement steps of the arrhythmia classification method using ECG signals described above.

In a fourth aspect, the present disclosure provides a computer-readable storage medium on which a computer program is stored, wherein the computer program, when executed by a processor, implements steps of the arrhythmia classification method using ECG signals described above.

In a fifth aspect, the present disclosure provides a computer program product, including a computer program, wherein the computer program, when executed by a processor, implements the steps of the arrhythmia classification method using ECG signals described above.

According to the specific embodiments provided by the present disclosure, the present disclosure discloses the following technical effects.

The present disclosure provides an arrhythmia classification method using ECG signals, an apparatus thereof, a device, a medium and a product. The method includes: acquiring Electrocardiograph (ECG) data and general patient data; constructing a first BERT model and a second BERT model; inputting the ECG data and the general patient data into the first BERT model and the second BERT model to obtain a first semantic information vector and a second semantic information vector; constructing a first tower layer and a second tower layer; inputting the first semantic information vector and the second semantic information vector into the first tower layer and the second tower layer, respectively, to obtain a first feature vector and a second feature vector; calculating a correlation coefficient based on the first feature vector and the second feature vector; carrying out feature combination on the first feature vector, the second feature vector and the correlation coefficient to obtain the combined feature; constructing a classification model; and inputting the combined feature into the classification model to obtain the category of arrhythmia classification. The present disclosure applies the ECG signals to the diagnosis and classification of arrhythmia for the first time, which greatly improves the accuracy of classification.

### BRIEF DESCRIPTION OF THE DRAWINGS

In order to explain the embodiments of the present disclosure or the technical solutions in the prior art more clearly, the drawings that need to be used in the embodiments will be briefly introduced hereinafter. Obviously, the drawings in the following description are only some embodiments of the present disclosure. For those skilled in the art, other drawings can be obtained according to these drawings without creative labor.

FIG. 1 is a flowchart of an arrhythmia classification method using ECG signals according to an embodiment of the present disclosure.

FIG. 2 is a flowchart block diagram of an arrhythmia classification method using ECG signals according to an embodiment of the present disclosure.

FIG. 3 is a schematic diagram of a 12-lead data channel relationship according to an embodiment of the present disclosure.

FIG. 4 is a diagram of the corresponding relationship between the components of ECG bands of a patient with arrhythmias and ECG signals in a twin-tower model according to an embodiment of the present disclosure.

FIG. 5A and FIG. 5B are schematic diagrams of formats of input data input1 and input2 according to an embodiment of the present disclosure.

FIG. 6 is a schematic structural diagram of a first tower layer according to an embodiment of the present disclosure.

FIG. 7 is a schematic structural diagram of a second tower layer according to an embodiment of the present disclosure.

FIG. 8 is a schematic diagram of an arrhythmia classification apparatus using ECG signals according to an embodiment of the present disclosure.

FIG. 9 is a schematic structural diagram of a computer device according to an embodiment of the present disclosure.

### DETAILED DESCRIPTION OF THE EMBODIMENTS

The technical solutions in the embodiments of the present disclosure will be clearly and completely described with reference to the drawings in the embodiments of the present disclosure hereinafter. Obviously, the described embodiments are only some embodiments of the present disclosure, rather than all of the embodiments. Based on the embodiments of the present disclosure, all other embodiments obtained by those skilled in the art without creative labor fall within the scope of protection of the present disclosure.

In order to make the above objects, features and advantages of the present disclosure more obvious and understandable, the present disclosure will be explained in further detail with reference to the drawings and embodiments hereinafter.

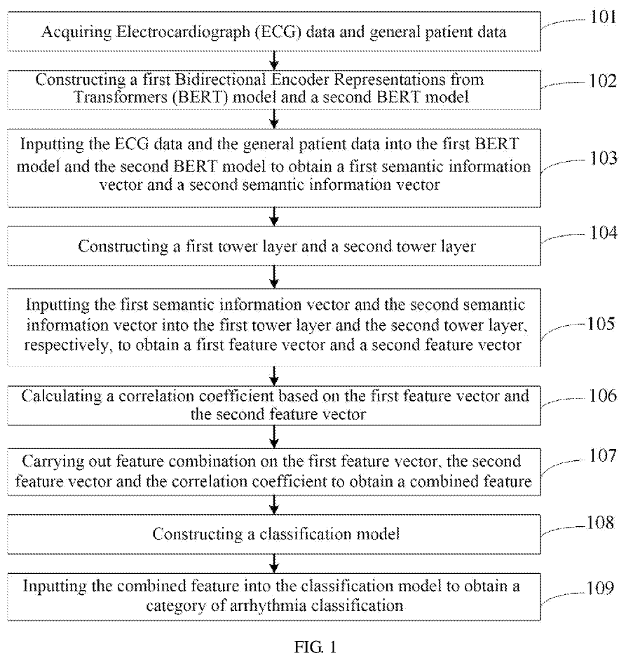

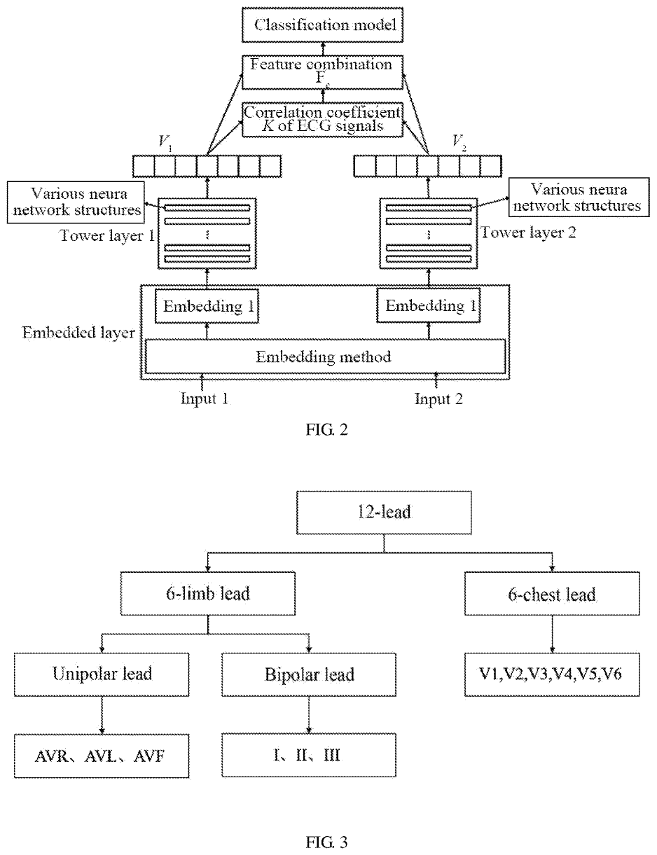

FIG. 1 is a flowchart of an arrhythmia classification method using ECG signals according to an embodiment of the present disclosure. FIG. 2 is a flowchart block diagram of an arrhythmia classification method using ECG signals according to an embodiment of the present disclosure. The method specifically includes steps 101-109.

Step 101: Electrocardiograph (ECG) data and general patient data are acquired.

ECG data is acquired by an ECG lead acquiring device or from an ECG database. The original device of acquiring ECG data includes 12-lead, in which the classification of leads and statistics of data dimensions are shown in FIG. 3.

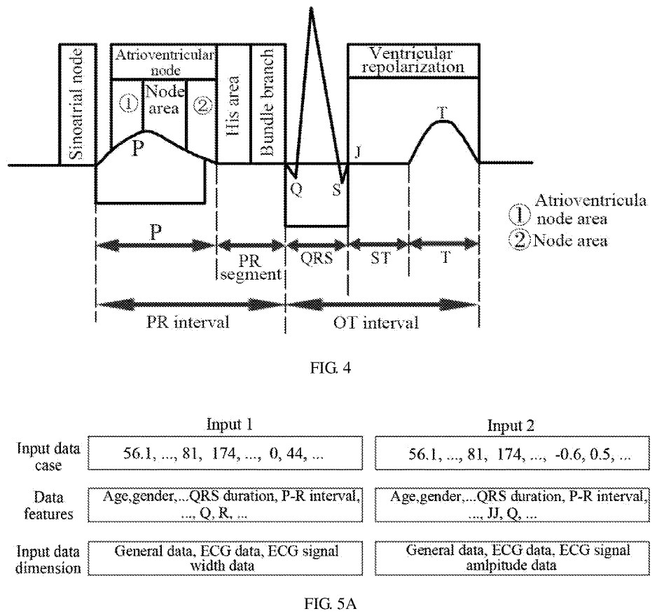

The general patient data includes but is not limited to an age, a sex, a height and a weight. Refer to Table 1 for details. Refer to FIG. 4 for ECG data, specifically including QRS duration (average QRS duration in milliseconds), P-R interval, P-T interval, T interval, P interval, QRS wave, T wave, P wave, QRST wave, J point, etc. Refer to Table 2 for details. ECG data further includes two sets of lead data distributions, namely, a wave width (refer to Table 3) and an amplitude (refer to Table 4). QRS wave represents ventricular depolarization complex, P wave represents the process of depolarization of the atria; Q wave represents initial negative deflection, R wave represents first positive deflection, S wave represents second negative deflection, T wave represents Ventricular repolarization wave, ST segment represents segment between ventricular depolarization and repolarization, J point represents the transition point between the completion of ventricular depolarization and the onset of repolarization.

TABLE 1
General data of patients with arrhythmia

| General features | Description of data features |
|---|---|
| Age | Linear |
| Sex | (0 = male; 1 = female), Nominal |
| Height | Height in centimeters, linear |
| Weight | Weight in kilograms, linear |
| . . . | . . . |

TABLE 2
General ECG data of patients with arrhythmia

| General ECG features | Description of data features |
|---|---|
| QRS duration | The average QRS duration in |
| | milliseconds reflects the changes of |
| | depolarization potential and time of left |
| | ventricle and right ventricle, Linear. |
| P-R interval | : the average duration between P wave |
| | and Q wave, Linear |
| Q-T interval | The average duration from the beginning |
| | of Q wave to the migration of T wave in |
| | milliseconds, Linear |
| T interval | The average duration of T wave in |
| | milliseconds, Linear |
| P interval | The average duration of P wave in |
| | milliseconds, Linear |
| QRS | |
| T | |
| P | |
| QRST | |
| J | A sudden turning point (junction) at the |
| | junction of the QRS complex and the ST |
| | segment on the ECG marks the end of |
| | ventricular depolarization and the |
| | beginning of repolarization |
| Heart rate | Heartbeats per minute, linear |
| . . . | . . . |

TABLE 3
Data of ECG signals of patients with
arrhythmia in wave width dimension

| Wave width features | Description | Lead |
|---|---|---|
| Q wave | Linear | Channel |
| R wave | Linear | DI |
| S wave | Linear | |
| R′ wave | Small peak just | |
| | after R, Linear | |
| S′ wave | Linear | |
| Number of intrinsic | Linear | |
| deflections | | |
| Existence of ragged R wave | Nominal | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | DII |
| S wave | Linear | |
| R′ wave | Small peak just | |
| | after R, Linear | |
| S′ wave | Linear | |
| Number of intrinsic | Linear | |
| deflections | | |
| Existence of ragged R wave | Nominal | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | DIII |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | AVR |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | AVL |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | AVF |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | V1 |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | V2 |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | V3 |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | V4 |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | V5 |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |
| Q wave | Linear | Channel |
| R wave | Linear | V6 |
| S wave | Linear | |
| R′ wave | Small peak just | |
| | after R, Linear | |
| S′ wave | Linear | |
| Number of intrinsic | Linear | |
| deflections | | |
| Existence of ragged R wave, | Nominal | |
| nominal | | |
| Existence of diphasic | Nominal | |
| derivation of R wave | | |
| Existence of ragged P wave | Nominal | |
| Existence of diphasic | Nominal | |
| derivation of P wave | | |
| Existence of ragged T wave | Nominal | |
| Existence of diphasic | Nominal | |
| derivation of T wave | | |

TABLE 4
Data of ECG signals of patients with
arrhythmia in amplitude dimension

| Amplitude features | Description of features | Lead |
|---|---|---|
| JJ wave | Linear | Channel |
| Q wave | Linear | DI |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | DII |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | DIII |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | AVR |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | AVL |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | AVF |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | V1 |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | V2 |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | V3 |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | V4 |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | V5 |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |
| JJ wave | Linear | Channel |
| Q wave | Linear | V6 |
| R wave | Linear | |
| S wave | Linear | |
| R′ wave | Linear | |
| S′ wave | Linear | |
| P wave | Linear | |
| T wave | Linear | |
| QRSA | The sum of the areas of all | |
| | line segments is divided by | |
| | 10 (area = width * | |
| | height/2), Linear | |
| QRSTA | QRSA + 0.5 * T wave width * | |
| | 0.1 * T wave height (If T is | |
| | biphasic, consider the larger | |
| | segment), Linear | |

Step 102: a first BERT model and a second BERT model are constructed.

In the present disclosure, the model in this step can also be a model or method with similar function to the BERT model, as long as the input data can be embedded, for example, DistilBERT, ROBERT (Robustly optimized BERT), ALBERT (A lite BERT), Word2Vec, etc.

Based on embeddable models or methods such as BERT, semantic feature embeddings are constructed using the general patient data and the ECG signal data. The BERT and other models support the models that can be embedded and effectively capture the contextual semantic features of the ECG signals, which are very sensitive to the complex semantic features of ECG in 12-lead and can understand the potential features between these digital signals.

Step 103: the ECG data and the general patient data are input into the first BERT model and the second BERT model to obtain a first semantic information vector and a second semantic information vector.

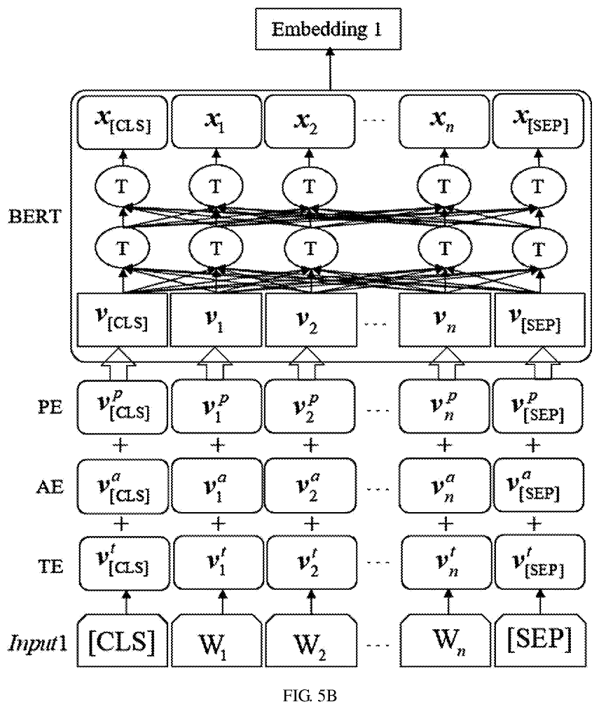

As shown in FIG. 5A and FIG. 5B, the data input format and the splicing method includes: general data+ECG data+ECG signal data (wave width), general data+ECG data +ECG signal data (amplitude).

Refer to FIG. 5A and FIG. 5B in detail, where PE represents position embedding, AE represents array block embedding and TE represents token embedding.

Step 104: a first tower layer and a second tower layer are constructed.

Specifically, the first tower layer and the second tower layer form a twin-tower model, and the first tower layer and the second tower layer share the same architecture and parameters. The design of the tower layer in the twin towers includes but is not limited to the neural network models related to deep learning such as LSTM, GRU, BiLSTM, BiGRU, CNN, RNN, Transformer and Attention, or the combined models of two or more thereof.

Step 105: the first semantic information vector and the second semantic information vector are input into the first tower layer and the second tower layer, respectively, to obtain a first feature vector and a second feature vector.

For example:

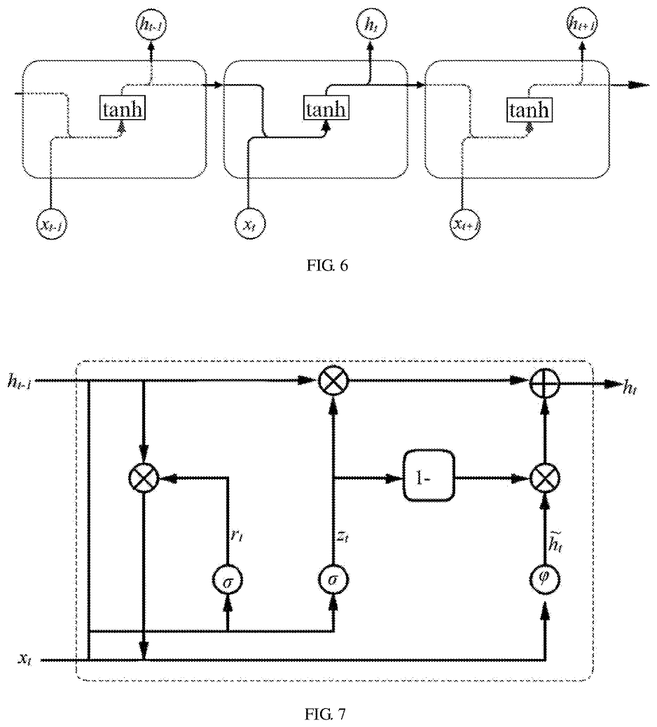

Refer to FIG. 6, which is a schematic structural diagram of the first tower layer. The RNN network structure model is taken as an example as the training model of the first tower layer. The RNN (Recurrent Neural Network) is a cyclic neural network, which is used to process the input1 data. The calculation process of the neural unit h₁ is as follows, and the output vector is V₁:

$$\text{?} = f ( {W}_{hh} {h}_{t - 1} + \text{?} {x}_{t} )$$

$$\text{?} \text{indicates text missing or illegible when filed}$$

where h is the feature vector denoting the moment t or t−1, x is the input vector of the input ECG signal, and W is the weight parameter.

$${V}_{1} = [ … , {h}_{t - 1} , {h}_{t} , \text{?} , … ]$$

$$\text{?} \text{indicates text missing or illegible when filed}$$

The GRU network structure model is taken as an example as the training model of the second tower layer.

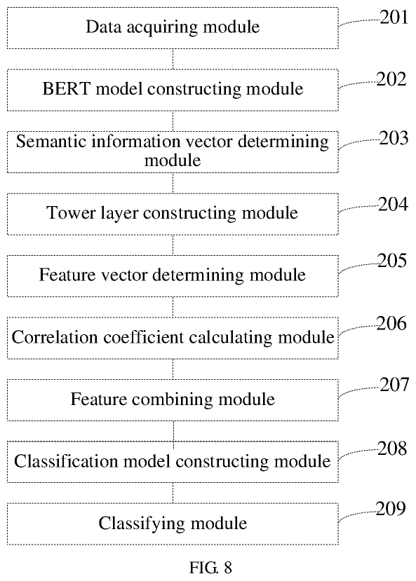

Refer to FIG. 7, which is the structural diagram of the GRU, which is used to calculate the input2 of the ECG signal. The calculation process of the neural unit h₁ is as follows, and the output vector is V₂:

$${z}_{t} = σ ( {W}_{z} [ {h}_{t - 1} , {x}_{t} ] )$$

$${z}_{t} = σ ( {W}_{r} [ {h}_{t - 1} , {x}_{t} ] )$$

$${\tilde{h}}_{t} = tanh ( W [ {r}_{t} * {h}_{t - 1} , {x}_{t} ] )$$

Where zₜ is the activation vector of the update gate, σ is sigmoid activation function, W_{z} is the weight parameter of the input xₜ, xₜ is the input vector of the ECG signal, rₜ is activation vector for the reset gate. Wᵣ is the weight matrix under the reset gate, \tilde{h}ₜ is the candidate hidden state at the current moment, tanh is the hyperbolic tangent function.

$${h}_{t} = ( 1 - {z}_{t} ) * {h}_{t - 1} + {z}_{t} * {\tilde{h}}_{t}$$

$${V}_{2} = [ … , {h}_{t - 1} , {h}_{t} , \text{?} , … ]$$

$$\text{?} \text{indicates text missing or illegible when filed}$$

Step 106: a correlation coefficient is calculated based on the first feature vector and the second feature vector.

The correlation coefficient K is calculated based on the two feature vectors with the same dimension generated based on the twin-tower model. The calculation methods of correlation coefficient K here include but are not limited to methods such as cosine similarity, Euclidean distance, Jaccard coefficient, Pearson correlation coefficient, Manhattan distance, Spearman's rank correlation coefficient, angular similarity, cosine distance and Chebyshev distance. The purpose of designing the correlation coefficient is to ensure the correlation between the whole data of the patient.

Taking cosine similarity as an example, the calculation process of the correlation coefficient is introduced in detail hereinafter.

V₁ and V₂ are two vectors with the same dimension, which are assumed to be n-dimensional. V₁ is denoted as: V₁={v₁, v₂, . . . , vₙ}, and is denoted as: V₂={μ₁, μ₂, . . . , μₙ}. The cosine value θ of the included angle between V₁ and V₂ is equal to:

$$cos θ = \frac{{\sum }_{i = 1}^{n} ( {v}_{i} \times {μ}_{i} )}{\sqrt{{\sum }_{i = 1}^{n} {( {v}_{i} )}^{2}} \times \sqrt{{\sum }_{i = 1}^{n} {( {μ}_{i} )}^{2}}} \qquad ( 1 )$$

$$cos θ = \frac{{V}_{1} {V}_{2}}{‖ {V}_{1} ‖ \times ‖ {V}_{2} ‖} \qquad ( 2 )$$

$$K = cos θ \qquad ( 3 )$$

The calculation of the cosine value θ is expressed as Formula 1 and Formula 2, in which Formula 3 is to calculate an assignment. The cosine value θ is expressed as the correlation coefficient of V₁ and V₁.

Step 107: feature combination is carried out on the first feature vector, the second feature vector and the correlation coefficient to obtain a combined feature.

Feature combination is carried out based on V₁, V₂ and K. The feature combination here includes but is not limited to the following two methods. In method 1, Concat operation between vectors is used to directly splice the features to be combined to obtain F_{C1}=[V₁, V₂, K], where the dimension of F_{C} is 1*(2m+1). In method 2, feature combination is carried out using the addition operation between vectors to obtain, F_{C2}=[V₁, V₂, K], where the feature dimension of F_{C} is 1*(m+1). In other methods, different calculation methods between vectors are used to obtain different feature combinations F_{C}. Although different methods for combining features are used, the methods are finally expressed as F_{C}.

Step 108: a classification model is constructed.

Based on F_{C}, the classification and diagnosis calculation of arrhythmia is carried out. The classification and diagnosis calculation methods here include but are not limited to related methods such as machine learning and deep learning. Further, the category of arrhythmia classification of patients with cardiovascular diseases includes normal, ischemic changes (coronary artery diseases), old forearm myocardial infarction, old lower arm myocardial infarction, sinus tachycardia, sinus bradycardia, premature ventricular contraction (PVC), ventricular premature contraction, left bundle branch block, right bundle branch block, 1-degree atrioventricular block, 2-degree atrioventricular block, 3-degree atrioventricular block, left ventricular hypertrophy, atrial fibrillation or flutter, etc.

Step 109: the combined feature is input into the classification model to obtain the category of arrhythmia classification.

Taking the support vector as an example, the classification and diagnosis calculation of arrhythmia based on F_{C} is explained in detail.

If the combined feature of the patient F_{C} is regarded as the feature vector of the patient, which is expressed as the vector X, all patients are represented as X, and the training data set T={(x₁, y₁), (x₂, y₂), . . . , (xₙ, yₙ)} of the patients with arrhythmia is constructed, where x₁∈ [symbol] ⁿ represents a combined feature vector of an i-th patient, yᵢ∈={1,2,3, . . . . M}, yi represents a classification category of the patient with arrhythmia, and M represents the number of categories of arrhythmia diseases. In addition, the values of i are 1, 2, . . . , N.

N samples related to arrhythmia are transformed from a low-dimensional space to a high-dimensional space. The samples are mapped into feature vectors in the high-dimensional space. Different sample data are separated according to the decision function ƒ(x), which maximizes the space interval between samples with various category labels. That is, N samples are divided into M categories. At the same time, the feature vector obtained by the space transformation of the sample X is expressed as φ(x), and the hyperplane Z corresponding to the feature vector φ(x) is:

$$\begin{matrix}( 4 ) & \\ f ( x ) = {ω}^{T} φ ( x ) + b & ( 4 )\end{matrix}$$

The objective function is:

$$min ( \frac{{‖ ω ‖}^{2}}{2} ) , s . t . {y}_{i} ( {ω}^{T} φ ( x ) + b ) \geq 1 , i = 1 , 2 , … , n \qquad ( 5 )$$

The Lagrange multiplier αi is used. Under the condition of constraint Σᵢαiyᵢ=0 and 0≤αi≤C, the formula is obtained:

$$max \text{?} - \frac{1}{2} \text{?} {\sum }_{j} \text{?} {y}_{i} {y}_{j} \text{?} {x}_{j} \qquad ( 6 )$$

$$s . t . \text{?} = 0 , i = 1 , 2 , … , n \qquad ( 7 )$$

$$\text{?} \text{indicates text missing or illegible when filed}$$

where j=1, 2, . . . , m, and i≠j. In order to improve the classification accuracy of categories of arrhythmia diseases, a kernel function K(xᵢ, yⱼ) is introduced to transform samples from a low-dimensional space to a high-dimensional space, transform the nonlinear multi-category problems into linear separable problems, and transform the samples X into φ(x) in a high-dimensional space, thus obtaining Formula (8).

$$K ( \text{?} ) = {φ ( \text{?} )}^{T} φ ( \text{?} ) \qquad ( 8 )$$

$$\text{?} \text{indicates text missing or illegible when filed}$$

The function of Formula (8) is substituted into Formula (6), and the arrhythmia classification function of the SVM model is calculated:

$$f ( x ) = {ω}^{T} φ ( x ) \text{?} b = sign ( \text{?} K ( \text{?} , {y}_{j} ) + b ) \qquad ( 9 )$$

$$\text{?} \text{indicates text missing or illegible when filed}$$

In addition, the kernel function required by the present disclosure include but are not limited to linear kernel function, polynomial kernel function, radial basis function and sigmoid kernel function. Through the arrhythmia classification function of Formula 9, classification and diagnosis can be carried out according to the combined feature F_{C} of the patient.

The present disclosure further provides an application scenario, which applies the arrhythmia classification method using ECG signals. Specifically, a model combining two or more neural networks is used to perform joint feature analysis on the two tower layers of the ECG signal data and to mine the relationship features with the types of arrhythmia diseases. Two models are calculated in the form of input constructed in the present disclosure (as shown in FIG. 3). Two vectors are given, and then the correlation is calculated to realize arrhythmia classification.

Based on the same inventive concept, the embodiment of the present disclosure further provides an arrhythmia classification apparatus using ECG signals for implementing the arrhythmia classification method using ECG signals. The solution to the problem provided by the apparatus is similar to that described in the above method. Therefore, the specific definitions of one or more embodiments of the arrhythmia classification apparatus using ECG signals provided hereinafter can be found in the above definition of the arrhythmia classification method using ECG signals provided above, which will not be described in detail here.

In an exemplary embodiment, as shown in FIG. 8, there is provided an arrhythmia classification apparatus using ECG signals, including:
a data acquiring module, configured to acquire Electrocardiograph (ECG) data and general patient data;
a BERT model constructing module, configured to construct a first BERT model and a second BERT model;
a semantic information vector determining module, configured to input the ECG data and the general patient data into the first BERT model and the second BERT model to obtain a first semantic information vector and a second semantic information vector;
a tower layer constructing module, configured to construct a first tower layer and a second tower layer;
a feature vector determining module, configured to input the first semantic information vector and the second semantic information vector into the first tower layer and the second tower layer, respectively, to obtain a first feature vector and a second feature vector;
a correlation coefficient calculating module, configured to calculate a correlation coefficient based on the first feature vector and the second feature vector;
a feature combining module, configured to carry out feature combination on the first feature vector, the second feature vector and the correlation coefficient to obtain a combined feature;
a classification model constructing module, configured to construct a classification model; and
a classifying module, configured to input the combined feature into the classification model to obtain a category of arrhythmia classification.

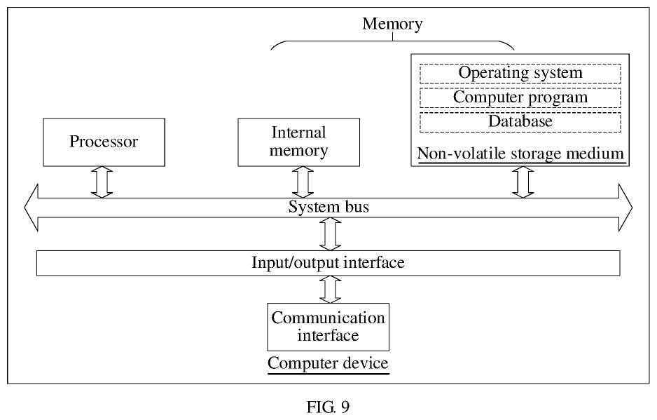

In an exemplary embodiment, a computer device is provided. The computer device may be a server or a terminal, the internal structure diagram of which may be as shown in FIG. 9. The computer device includes a processor, a memory, an Input/Output interface (I/O for short) and a communication interface. The processor, the memory and the input/output interface are connected through a system bus, and the communication interface is connected to the system bus through the input/output interface. The processor of the computer device is configured to provide computing and control capabilities. The memory of the computer device includes a non-volatile storage medium and an internal memory. The non-volatile storage medium stores an operating system, a computer program and a database. The internal memory provides an environment for the operation of the operating system and the computer program in the non-volatile storage medium. The database of the computer device is configured to store arrhythmia classification data using ECG signals. The input/output interface of the computer device is configured to exchange information between the processor and the external device. The communication interface of the computer device is configured to be communicated with an external terminal through network connection. The computer program, when executed by the processor, implements an arrhythmia classification method using ECG signals.

It can be understood by those skilled in the art that the structure shown in FIG. 9 is only a block diagram of a part of the structure related to the solution of the present disclosure, and does not constitute a limitation on the computer device to which the solution of the present disclosure is applied. The specific computer device may include more or less components than those shown in the figure, or combine some components, or have different component arrangements.

In an exemplary embodiment, a computer device is further provided, which includes a memory and a processor, wherein a computer program is stored in the memory, and the processor, when executing the computer program, implements the steps in the above method embodiments.

In an exemplary embodiment, a computer-readable storage medium is provided, on which a computer program is stored, wherein the computer program, when executed by a processor, implements the steps in the above method embodiments.

In an exemplary embodiment, a computer program product is provided, including a computer program, wherein the computer program, when executed by a processor, implements the steps in the above method embodiments.

It should be noted that the user information (including but not limited to user equipment information, user personal information, etc.) and data (including but not limited to data for analysis, stored data, displayed data, etc.) involved in the present disclosure are all information and data authorized by users or fully authorized by all parties, and the collection, use and processing of relevant data should comply with relevant regulations.

Those skilled in the art can understand that all or part of the processes in the method of implementing the above embodiment can be completed by instructing related hardware through a computer program. The computer program can be stored in a non-volatile computer-readable storage medium, and when executed, the computer program can include the processes of the embodiments of the above method. Any reference to the memory, the database or other media used in various embodiments provided by the present disclosure may include at least one of a non-volatile memory and a volatile memory. The non-volatile memory may include a Read-Only Memory (ROM), a magnetic tape, a floppy disk, a flash memory, an optical memory, a high-density embedded non-volatile memory, a Resistive Random Access Memory (ReRAM), a Magneto-Resistive Random Access Memory (MRAM), a Ferroelectric Random Access Memory (FRAM), a Phase Change Memory (PCM), a graphene memory, etc. The volatile memory may include a Random Access Memory (RAM) or an external cache memory. By way of illustration and not limitation, RAM can be in various forms, such as a Static Random Access Memory (SRAM) or a Dynamic Random Access Memory (DRAM).

The databases involved in various embodiments provided by the present disclosure may include at least one of a relational database and a non-relational database. The non-relational

What is claimed is:

1. An arrhythmia classification method using ECG signals, wherein the arrhythmia classification method using the ECG signals comprises:
acquiring Electrocardiograph (ECG) data and general patient data;
constructing a first Bidirectional Encoder Representations from Transformers (BERT) model and a second BERT model;
inputting the ECG data and the general patient data into the first BERT model and the second BERT model to obtain a first semantic information vector and a second semantic information vector;
constructing a first tower layer and a second tower layer;
inputting the first semantic information vector and the second semantic information vector into the first tower layer and the second tower layer, respectively, to obtain a first feature vector and a second feature vector;
calculating a correlation coefficient based on the first feature vector and the second feature vector;
carrying out feature combination on the first feature vector, the second feature vector and the correlation coefficient to obtain a combined feature;
constructing a classification model; and
inputting the combined feature into the classification model to obtain a category of arrhythmia classification.

2. The arrhythmia classification method using the ECG signals according to claim 1, wherein the general patient data comprises an age, a sex, a height and a weight;
the ECG data comprises general ECG data and lead data distribution, the general ECG data comprises QRS duration, P-R interval, P-T interval, T interval, P interval, QRS wave, T wave, P wave, QRST wave and J point; and the lead data distribution comprises a wave width and an amplitude.

3. The arrhythmia classification method using the ECG signals according to claim 1, wherein the first tower layer is any one of Long Short-Term Memory (LSTM), Gate Recurrent Unit (GRU), Bidirectional Long Short-Term Memory (BiLSTM), Bidirectional Gate Recurrent Unit (BiGRU), Convolutional Neural Network (CNN), Recurrent Neural Network (RNN), Transformer and Attention;
the second tower layer is any one of LSTM, GRU, BILSTM, BIGRU, CNN, RNN, Transformer and Attention.

4. The arrhythmia classification method using the ECG signals according to claim 1, wherein the correlation coefficient is calculated based on the first feature vector and the second feature vector using any one of methods comprising cosine similarity, Euclidean distance, Jaccard similarity coefficient, Pearson correlation coefficient, Manhattan distance, Spearman's rank correlation coefficient, angular similarity, cosine distance and Chebyshev distance.

5. The arrhythmia classification method using the ECG signals according to claim 1, wherein the correlation coefficient is calculated based on the first feature vector and the second feature vector using a following formula:
$$cos θ = \frac{{\sum }_{i = 1}^{n} ( {v}_{i} \times {μ}_{i} )}{\sqrt{{\sum }_{i = 1}^{n} {( {v}_{i} )}^{2}} \times \sqrt{{\sum }_{i = 1}^{n} {( {μ}_{i} )}^{2}}} or cos θ = \frac{{V}_{1} {V}_{2}}{‖ {V}_{1} ‖ \times ‖ {V}_{2} ‖}$$
$$K = cos θ$$
wherein V₁={v₁, v₂, . . . , vₙ} represents the first feature vector, and V₂={μ₁, μ₂, . . . , μₙ} represents the second feature vector.

6. The arrhythmia classification method using the ECG signals according to claim 1, wherein an expression of the classification model is:
$$f ( x ) = {ω}^{T} φ ( x ) \text{?} b = sign ( \text{?} K ( {x}_{i} , {y}_{j} ) + b )$$
$$\text{?} \text{indicates text missing or illegible when filed}$$
where ω represents a parameter matrix, i.e. a weight vector, T represents transposition calculation, φ(x) represents a nonlinear mapping function that maps input features into a high-dimensional space, b represents a bias term, n represents a number of patients, i represents an i-th patient, yᵢ represents a classification category of the i-th patient with arrhythmia, yⱼ represents a classification category of a j-th patient with arrhythmia, αᵢ represents a Lagrange multiplier corresponding to an i-th training sample, and K(xᵢ, yⱼ) represents a kernel function, which is used to calculate similarity between input features.

7. An arrhythmia classification apparatus using ECG signals, wherein the arrhythmia classification apparatus using the ECG signals comprises:
a data acquiring module, configured to acquire Electrocardiograph (ECG) data and general patient data;
a BERT model constructing module, configured to construct a first BERT model and a second BERT model;
a semantic information vector determining module, configured to input the ECG data and the general patient data into the first BERT model and the second BERT model to obtain a first semantic information vector and a second semantic information vector;
a tower layer constructing module, configured to construct a first tower layer and a second tower layer;
a feature vector determining module, configured to input the first semantic information vector and the second semantic information vector into the first tower layer and the second tower layer, respectively, to obtain a first feature vector and a second feature vector;
a correlation coefficient calculating module, configured to calculate a correlation coefficient based on the first feature vector and the second feature vector;
a feature combining module, configured to carry out feature combination on the first feature vector, the second feature vector and the correlation coefficient to obtain a combined feature;
a classification model constructing module, configured to construct a classification model; and
a classifying module, configured to input the combined feature into the classification model to obtain a category of arrhythmia classification.

8. A computer device, comprising: a memory, a processor and a computer program which is stored in the memory and is executable on the processor, wherein the processor executes the computer program to implement steps of the arrhythmia classification method using the ECG signals according to any one of claim 1.

9. A non-transitory computer-readable storage medium on which a computer program is stored, wherein the computer program, when executed by a processor, implements steps of the arrhythmia classification method using the ECG signals according to any one of claim 1.
